(19) United States
(12) Patent Application Publication
(10) Pub. No.: US 2010/0043915 A1
(43) Pub. Date: Feb. 25, 2010

(54) ASEPTIC BEVERAGE BOTTLE FILLING PLANT WITH A CLEAN ROOM ARRANGEMENT ENCLOSING THE ASEPTIC BEVERAGE BOTTLE FILLING PLANT AND A METHOD OF OPERATING SAME, AND AN ASEPTIC CONTAINER FILLING PLANT WITH A CLEAN ROOM ARRANGEMENT ENCLOSING THE ASEPTIC CONTAINER FILLING PLANT, AND A METHOD OF OPERATING SAME

(76) Inventors: Daryoush Sangi, Hamburg (DE); Thomas Herold, Ahrensburg (DE)

Correspondence Address: NILS H. LJUNGMAN & ASSOCIATES, P. O. BOX 130, GREENSBURG, PA, 15601-0130, US

(21) Appl. No.: 12/362,633

(22) Filed: Jan. 30, 2009

Related U.S. Application Data
(63) Continuation-in-part of application No. PCT/EP2007/006805, filed on Aug. 1, 2007.

(30) Foreign Application Priority Data
Aug. 4, 2006 (DE) 10 2006 036 475.9

(51) Int. Cl. B65B 1/04; B08B 9/00

(52) U.S. Cl. 141/11; 141/92; 134/22.1

(57) ABSTRACT

An aseptic beverage bottle filling plant with a clean room arrangement enclosing the aseptic beverage bottle filling plant and a method of operating same, and an aseptic container filling plant with a clean room arrangement enclosing the aseptic container filling plant, and a method of operating same. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/EP2007/006805, filed on Aug. 1, 2007, which claims priority from Federal Republic of Germany Patent Application No. 10 2006 036 475.9, filed on Aug. 4, 2006. International Patent Application No. PCT/EP2007/006805 was pending as of the filing date of this application. The United States was an elected state in International Patent Application No. PCT/EP2007/006805.

### BACKGROUND

1. Technical Field

The present application relates to an aseptic beverage bottle filling plant with a clean room arrangement enclosing the aseptic beverage bottle filling plant and a method of operating same, and an aseptic container filling plant with a clean room arrangement enclosing the aseptic container filling plant, and a method of operating same.

2. Background Information

Background information is for informational purposes only and does not necessarily admit that subsequently mentioned information and publications are prior art.

Aseptic techniques are becoming increasingly important for the handling and filling of containers, such as with beverages or medicines. To achieve a long shelf life of the contents of the container without the use of chemical preservatives or other preservation methods, such as heat treatment, etc., it is of decisive importance that both the containers themselves as well as the handling, filling or other systems that come in contact with the containers are as germ-free as possible. To this end, the handling or filling devices are enclosed in a housing and placed under positive pressure, and measures such as the use of air filters, airlocks, etc. are taken to essentially ensure or promote that only germ-free materials can gain admission to the interior.

To essentially ensure or promote an environment that is as germ-free as possible, the interior of such machines must or should also be cleaned periodically. A variety of methods are used. Typically, a chemical sterilizing medium is dispensed during a sterilization cycle into the interior of the machine via sprayer heads installed in the interior, where this medium reacts with any undesired germs, etc. present and kills them. Cleaning the interior as thoroughly as possible requires or desires a plurality of sprayer heads so that all areas of the in part complexly structured apparatus in the interior of the machine can be cleaned effectively. One such sterilizing medium used is peracetic acid.

Disadvantageous with this type of cleaning is that a plurality of sprayer heads must be arranged in the interior of the machine to enable complete or virtually complete wetting of all surfaces with the disinfectant. Furthermore, the efficacy of the liquid sterilizing media sometimes used is inadequate, or these media are not approved for food applications.

### OBJECT OR OBJECTS

An object of the present application is to provide a cleaning and/or sterilization method that enables or promotes effective cleaning and/or sterilization of the interior of the clean rooms without having to install additional costly and complex cleaning systems.

### SUMMARY

The present application relates to a clean room sterilization arrangement, and in one possible embodiment to a clean room and method of cleaning that clean room for subsequent handling and/or filling and closing of containers for relatively germ free production and filling of those containers.

The interior of clean rooms for such machines according to the present application also includes a device for sterilizing the containers to be handled and/or filled using a vaporous sterilizing medium. The sterilizing medium is directed to at least the interior and the upper edge region of the container, where it precipitates and forms a condensation film due to the cooler container surface temperature relative to the sterilizing medium. A certain amount of heat that can activate the medium is then added to the sterilizing medium, so that the actual sterilization reaction can then occur. If hydrogen peroxide (H₂O₂) is used as the sterilizing medium, a decomposition reaction is initiated by the addition of heat, for example, in which reaction the H₂O₂ decomposes and the decomposition products contain free radicals that react with the germs and other impurities present. At the end of the reaction, essentially all that remains is water and possibly some few decomposition products, whereby in one possible embodiment no harmful germs, etc. are present, so a degree of sterility satisfying the requirements for food or pharmaceutical applications is achieved.

The present application teaches that the equipment for introducing the vaporous sterilizing medium into the containers are also used to sterilize the entire machine. This is done by discharging the vaporous sterilization medium, for example H₂O₂, into the atmosphere, which becomes enriched with the medium, via the sterilization system during idle mode operation without containers. Exactly as in the containers, the medium precipitates onto all surfaces with which it comes in contact due to the differential temperatures and forms a condensation film there. The discharged vaporous medium is dispersed throughout the entire machine by means of appropriate air routing. Various sequences can be used depending on the configuration of the machine. The doors present between individual sections of the machine can be used to control where and in what amount the vaporous medium is to be transported. This is supported by the controlled admission of purified fresh air, which entrains the vaporous medium into the various sections of the machine. Analogously, installed extraction devices can generate an air flow in the interior of the machine that carries the vaporous sterilizing medium to the various sections of the machine where it can form a condensation film as described above.

Depending on the machine, it can be appropriate to evenly fill the entire machine with a sufficient concentration of the vaporous sterilizing media or to fill various sections of the machine sequentially.

As soon as a sufficient quantity of sterilizing medium is distributed in the interior of the machine, the medium can be activated by heat in the event that H₂O₂ is used. This can be done via the admission of hot steam or hot air. The machine can be equipped for this purpose with air supply and filtration devices with integrated heating devices, which devices introduce the appropriate quantities of hot, filtered air into the interior of the clean room and thus initiate the decomposition reaction of the sterilizing medium. Here, too, the reaction essentially can be initiated simultaneously or substantially simultaneously in all locations via the simultaneous or substantially simultaneously admission of sufficient quantities of hot air in all sections of the machine, or alternatively activation can occur in steps from section to section.

Once the reaction has ended, the installed extraction devices can be used to extract any residual reaction products from the interior of the machine. Targeted air routing is used here as well to essentially ensure or promote that all decomposition products are removed as thoroughly as possible and that the interior air is exchanged, if necessary and/or desired.

Depending on the design of the machine, piping present can be sterilized similarly. Here the vaporous sterilizing media is pumped through the piping and activated by hot air or hot steam. Alternatively, a mixture of the vaporous sterilizing medium and hot steam can be passed directly through the pipes. This step can be performed prior to the described sterilization of the interior, since then any decomposition products discharged from the piping are removed with the remaining decomposition products from the machine.

Depending on the machine, it may be necessary or desired to have activation devices immediately or generally downstream of the container sterilization devices, which devices activate the condensation film applied to the inside of the containers by the admission of hot steam or hot air, for example, during normal operation. In one embodiment, these can also be used in sterilization mode to dispense the vaporous sterilizing medium into the interior of the machine, which can significantly or somewhat accelerate the method.

These and other embodiments of the present application are disclosed according to the present application, which are described below.

The present application also relates to a cleaning machine for the sterilization of clean rooms for the handling and/or filling of containers using the method described and also a control device for performing the method described. Such a cleaning device and such a control device also solve the defined task.

The present application thus comprises a beverage container filling plant for filling containers with a fluid, comprising: a sealable housing enclosure enclosing a series of clustered container treating mechanisms, wherein the housing enclosure comprises a clustered arrangement of chambers comprising: a first chamber, a second chamber, a third chamber and a fourth or container filling and sealing chamber, each chamber arranged adjacent to one another in a generally circular pattern, to permit sequentially processing containers passing through the cluster of mechanisms; a first conduit system for sequentially or simultaneously discharging cleansing fluid into the first chamber, the second chamber, the third chamber and the fourth or container filling chamber, the system for discharging cleansing fluid comprising a plurality of discharge heads arranged within each of the chambers for spraying the cleansing fluid onto the containers and/or onto the cluster of container support mechanisms within the cluster of chambers; a second conduit system for effecting a positive pressure fluid within the housing enclosure, the second cleansing system comprising a plurality of discharge vents disposed within the second chamber, the third chamber and the fourth or container filling chamber, the positive pressure fluid comprising a pressurized hot gaseous fluid from a discharge vent to fill the enclosure during a treatment cycle; a container entry port with an openable and closable door for initial feeding of containers into the first chamber of the clustered housing enclosure; a plurality of star wheel rotary tables for receiving and transporting the containers from the container entry port through the process occurring in the first chamber and subsequently into the second chamber; a plurality of star wheel rotary tables for receiving and transporting the containers from the process occurring in the first chamber through the cleansing process occurring in the second chamber and subsequently transporting the containers into the third chamber; a plurality of star wheel rotary tables within the third chamber for receiving and transporting the containers from the cleansing process occurring in the second chamber and through the cleansing process occurring in the third chamber and subsequently transporting the containers into the fourth or container filling chamber; a plurality of star wheel rotary tables within the fourth or container filling chamber for receiving and transporting the containers from the cleansing process occurring in the third chamber and onto a rotary table filling station through the container filling process occurring in the fourth or container filling chamber and a further plurality of star wheel rotary tables within the fourth or container filling chamber for subsequently transporting filled containers into the container closing device; a container alignment track for directing filled containers away from the container closing device and out of the enclosure housing through a filled container discharge port; and an extraction system arranged within the container entry port and the filled container discharge port for withdrawing the positively pressurized hot gaseous fluid through the machine and simultaneously or substantially simultaneously out through both the entry port and the filled container discharge port. The container filling chamber may have a discharge head arranged over the rotary table filling station for cleansing the rotary filing table during a cleansing cycle of the rotary filing table. The containers passing through the first chamber may be treated by a medium comprising vaporous hydrogen peroxide. The medium treated containers may have a film of the medium on the containers passing through the second chamber and the third chamber, and are then treated with a hot treatment medium. The hot treatment medium is in one possible embodiment comprised of heated air and/or steam. The first conduit system may be sterilized and cleaned by the second conduit system during its utilization. The conduits of the second conduit system may be sterilized and cleaned by the second conduit system during its utilization. A container conveyor mechanism is in one possible embodiment arranged between the rotary table arrangement in the first chamber and the second chamber to transfer the containers from the first chamber to the second chamber. A container conveyor mechanism is in one possible embodiment arranged between the rotary table arrangement in the second chamber to transfer the containers from the second chamber to the rotary table arrangement in the third chamber. A container conveyor mechanism is in one possible embodiment arranged between the rotary table arrangement in the third chamber and the rotary table arrangement in the container filling chamber to transfer the containers from the third chamber to the rotary table arrangement in the fourth or container filling chamber.

The present application may also include a method of establishing and operating a beverage container filling plant, comprising one or more of the following: arranging a sealable housing enclosure for enclosing a series of clustered container treating mechanisms into a clustered generally circular arrangement of generally adjacent chambers comprising: a first chamber, a second chamber, a third chamber and a fourth or container filling and sealing chamber, each chamber arranged next to one another for sequentially processing containers passing through the cluster of mechanisms; connecting a first conduit system for sequentially or simultaneously discharging cleansing fluid into the first chamber, the second chamber, the third chamber and the fourth or container filling chamber, wherein the system for discharging cleansing fluid comprises a plurality of discharge heads arranged within each of the chambers for spraying the cleansing fluid onto the containers and/or onto the cluster of container support mechanism within the cluster of chambers; arranging a second conduit system for effecting a positive pressure fluid within the housing enclosure, wherein the second conduit system comprises a plurality of discharge vents disposed within the second chamber, the third chamber and the fourth or container filling chamber, and wherein the positive pressure fluid comprises a pressurized hot gaseous fluid from a discharge vent to fill the enclosure during a treatment cycle; placing a container entry port with an openable and closable door for initial feeding of containers into the first chamber of the clustered housing enclosure; arranging a plurality of star wheel rotary tables for receiving and transporting the containers from the container entry port through the process occurring in the first chamber and subsequently into the second chamber; arranging a plurality of star wheel rotary tables for receiving and transporting the containers from the process occurring in the first chamber through the cleansing process occurring in the second chamber and subsequently transporting the containers into the third chamber; arranging a plurality of star wheel rotary tables within the third chamber for receiving and transporting the containers from the cleansing process occurring in the second chamber and through the cleansing process occurring in the third chamber and subsequently transporting the containers into the fourth or container filling chamber; arranging a plurality of star wheel rotary tables within the fourth or container filling chamber for receiving and transporting the containers from the cleansing process occurring in the third chamber and onto a rotary table filling station through the container filling process occurring in the fourth or container filling chamber and a further plurality of star wheel rotary tables within the fourth or container filling chamber for subsequently transporting the now filled containers into the container closing device; arranging a container alignment track for directing filled containers away from the container closing device and out of the enclosure housing through a filled container discharge port; and installing an extraction system within the container entry port and the filled container discharge port for withdrawing the positively pressurized hot gaseous fluid through the machine and simultaneously or substantially simultaneously out through both the entry port and the filled container discharge port. The first chamber comprises a sterilization chamber, the second chamber comprises a first activation chamber, and the third chamber comprises a second activation chamber.

The present application also comprises a process for cleaning a beverage container filling plant, which plant is used for filling containers with a fluid, comprising one or more of the following: arranging a sealable housing enclosure enclosing a series of clustered container treating mechanisms, wherein the housing enclosure comprises a clustered arrangement of chambers comprising: a first chamber, a second chamber, a third chamber and a fourth or container filling and sealing chamber, each chamber arranged closely adjacent one another for sequentially processing containers passing through the cluster of mechanisms; introducing a cleansing fluid through a first conduit system for sequentially or simultaneously discharging the cleansing fluid into the first chamber, the second chamber, the third chamber and the fourth or container filling chamber, the system for discharging cleansing fluid comprising a plurality of discharge heads arranged within each of the chambers for spraying the cleansing fluid onto the containers and/or onto the cluster of container support mechanism within the cluster of chambers; introducing a positively pressurized fluid through a second conduit system within the housing enclosure, which second conduit system comprises a plurality of discharge vents disposed within the second chamber, the third chamber and the fourth or container filling chamber, the positive pressure fluid comprising a pressurized hot gaseous fluid to fill the housing enclosure during a treatment or machine cleansing cycle; opening or closing an openable and closable door on a container entry port to permit initial feeding or denial of containers into the first chamber of the clustered housing enclosure; directing containers about a plurality of star wheel rotary tables for transporting the containers from the container entry port through the process occurring in the first chamber and subsequently into the second chamber; arranging a plurality of star wheel rotary tables for receiving and transporting the containers from the process occurring in the first chamber through the cleansing process occurring in the second chamber and subsequently transporting the containers into the third chamber; arranging a plurality of star wheel rotary tables within the third chamber for receiving and transporting the containers from the cleansing process occurring in the second chamber and through the cleansing process occurring in the third chamber and subsequently transporting the containers into the fourth or container filling chamber; arranging a plurality of star wheel rotary tables within the fourth or container filling chamber for receiving and transporting the containers from the cleansing process occurring in the third chamber and onto a rotary table filling station through the container filling process occurring in the fourth or container filling chamber and a further plurality of star wheel rotary tables within the fourth or container filling chamber for subsequently transporting the now filled containers into the container closing device; placing a container alignment track for directing filled containers away from the container closing device and out of the enclosure housing through a filled container discharge port; and routing positively pressurized hot gaseous fluid out of the machine through an extraction system arranged within both the container entry port and the filled container discharge port simultaneously or substantially simultaneously. The fourth or container filling chamber may have a discharge head arranged over the rotary table filling station for cleansing the rotary filing table during a cleansing cycle. The containers passing through the first chamber may be treated by a sterilizing medium comprising vaporous hydrogen peroxide. The containers treated with the film of the sterilizing medium on the containers passing through the second chamber and the third chamber may be treated with a hot gaseous treatment medium. The treatment medium may be comprised of heated air and/or steam. The first conduit system is in one possible embodiment sterilized and cleaned by the second conduit system during its utilization. The conduits of the second conduit system are in one possible embodiment sterilized and cleaned by the second conduit system during its utilization.

The present application also comprises a process for operating an aseptic beverage container filling plant, comprising one or more of the following: arranging a generally circular array of chambers into a series of container treatment mechanisms, and forming the beverage container filling plant comprising a sterilization chamber, a first activation chamber, a second activation chamber and a container filling chamber, the sterilization chamber comprising a container entry port and the container filling chamber having a container discharge port; connecting the chambers with container transport apparatus for moving treated containers therebetween; installing a plurality of fluid discharge heads within more than one of the chambers, and connecting the discharge heads to a fluid supply circuit; installing a plurality of vent units for the discharge of a second fluid within the activation chambers and the container filling chamber; installing partition doors within the beverage container filling plant configured to control the flow of any treatment fluid and any activation fluid within the chambers of the filling plant; applying a treatment fluid within the sterilization chamber through the fluid discharge heads therein; distributing treatment fluid through the generally circular array of chambers by activating a first extraction device configured to move the treatment fluid through all the chambers; delivering an activation gas through the vent units at a general mid-point of a process path through which the containers travel within the container filling plant; activating the treatment fluid dispersed onto the container treatment mechanisms; activating a second gas extraction device to permit the movement of the gas and activation fluid out both an open first and an open second port which first and second ports are generally adjacent one another, to thus permit the bottle filling plant to be cleansed by controlled fluid routing, and thus exhausting the activation fluid and the gas from both the first and second port through the shortest most direct routes out of the beverage container filling plant, to impede entry of germs into the filling plant, therefore minimizing travel distance and maintaining maximum effectiveness of treatment activity of treatment gases and thus readying the beverage container filling plant for a container filling cycle; delivering a plurality of empty containers into the first port in the first or sterilization chamber; directing a treatment fluid onto the empty containers within the first or sterilization chamber; moving the empty containers through the container process path for their subsequent activation treatment in the second chamber and the third chamber; applying an activation fluid onto the empty containers during their travel on the process path through the second and third activation chambers; directing the now-sterilized empty containers into the filling chamber for subsequent filling of the containers with a consumable beverage; filling the empty containers located within the filling chamber with a consumable beverage; closing the filled containers and directing those containers out of the filling plant on a filled-container discharge track; and discharging the now-filled containers out of the beverage container filling plant through the second port, thus completing the generally circular route of the beverage containers through the beverage container filling plant. The process may include: introducing treatment fluid through the discharge heads within the activation chambers and the container filling chamber, so as to provide the treatment fluid for cleansing the container treatment mechanisms therein, and wherein the treatment fluid may comprise hydrogen peroxide, and wherein the gas delivered through the vent units to activate the treatment fluid may be a heated gas, and wherein the gas delivered to the filling chamber may be a purified gas.

The present application may also comprise an apparatus for performing the process of operating an aseptic beverage container filling plant, the apparatus having means comprising: an isolated generally circular array of chambers into a series of container treatment mechanisms, for the forming of the beverage container filling plant comprising a sterilization chamber, a first activation chamber, a second activation chamber and a container filling chamber, the sterilization chamber comprising a container entry port and the container filling chamber having a container discharge port, the chambers being connected with container transport lines for moving treated containers therebetween; a plurality of fluid discharge heads arranged within more than one of the chambers, the discharge heads being connected to a fluid supply circuit; a plurality of vent units arranged for the discharge of a second fluid within the activation chambers and the container filling chamber; an arrangement of partition doors within the beverage container filling plant configured to control the flow of any treatment fluid and any activation fluid within the chambers of the filling plant; a treatment fluid discharged within the sterilization chamber through the fluid discharge heads therein, the treatment fluid distributed through the generally circular array of chambers by activating a first extraction device configured to move the treatment fluid through all the chambers; a gas delivered through the vent units at a general mid-point of a process path through which the containers travel within the container filling plant, and wherein the treatment fluid is dispersed onto the container treatment mechanisms; a second gas extraction device being arranged to move the gas and activation fluid out an open first and an open second port which ports are generally adjacent one another, to thus permit the bottle filling plant to be cleansed by controlled fluid routing, and thus exhausting the activation fluid and the gas from both the first and second port through the shortest most direct routes out of the beverage container filling plant to impede entry of germs into the filling plant, therefore minimizing travel distance of treatment gases thus readying the beverage container filling plant for a container filling cycle, and wherein a plurality of empty containers are arranged for delivery into the first port in the first or sterilization chamber; a treatment fluid for discharge onto the empty containers within the first or sterilization chamber, wherein the empty containers are moved through the container process path for their subsequent activation treatment in the second chamber and the third chamber; an activation fluid application onto the empty containers during their travel on the process path through the second and third activation chambers; wherein the now-sterilized empty containers are directed into the filling chamber for subsequent filling of the containers with a consumable beverage, and filling the empty containers located within the filling chamber with a consumable beverage; a closing means for the filled containers and a directing means for those containers out of the filling plant on a filled-container discharge track; and a discharge means for moving the now-filled containers out of the beverage container filling plant through the second port, thus completing the generally circular route of the containers through the beverage container filling plant. The apparatus may include: a heating means arranged within the vent units, the heating means arranged to controllably heat fluid passing therethrough.

The present application may also comprise a method for operating an aseptic beverage container filling plant, comprising one or more of the following: connecting an array of chambers into a series of container treatment mechanisms, and forming the beverage container filling plant from those chambers which together comprise a sterilization chamber, a first activation chamber, a second activation chamber and a container filling chamber, the sterilization chamber comprising a container entry port and the container filling chamber having a container discharge port; connecting the chambers with container transport apparatus for moving treated containers therebetween; installing a plurality of fluid discharge heads within more than one of the chambers, and connecting the discharge heads to a fluid supply circuit; installing a plurality of vent units for the discharge of a second fluid within the activation chambers and the container filling chamber; installing partition doors within the beverage container filling plant, the doors configured to control the flow of any treatment fluid and any activation fluid within the chambers of the filling plant; applying a treatment fluid within the sterilization chamber through the fluid discharge heads therein; distributing treatment fluid through the array of chambers by activating a first extraction device configured to move the treatment fluid through all the chambers; delivering an activation gas through the vent units located adjacent the process path through which the containers travel within the container filling plant; activating the treatment fluid dispersed onto the container treatment mechanisms; activating a second gas extraction device to permit the movement of the gas and activation fluid out multiple ports which ports are generally adjacent one another, to thus permit the bottle filling plant to be cleansed by controlled fluid routing, and thus removing the activation fluid and the gas from both the multiple ports through the shortest most direct routes out of the beverage container filling plant, to impede entry of germs into the filling plant, therefore minimizing travel distance and maintaining maximum effectiveness of treatment activity of treatment gases and thus readying the beverage container filling plant for a container filling cycle; delivering a plurality of empty containers into the first port in the first or sterilization chamber; directing a treatment fluid onto the empty containers within the first or sterilization chamber; moving the empty containers through the container process path for their subsequent activation treatment in the second chamber and the third chamber; applying an activation fluid onto the empty containers during their travel on the process path through the second and third activation chambers; directing the now-sterilized empty containers into the filling chamber for subsequent filling of the containers with a consumable beverage; filling the empty containers located within the filling chamber with a consumable beverage; closing the filled containers and directing those containers out of the filling plant on a filled-container discharge track; and discharging the now-filled containers out of the beverage container filling plant through the second port, thus completing the process path route of the beverage containers through the beverage container filling plant. The process path of the containers within the beverage processing plant is in one possible embodiment of generally circular configuration. The activation gas may be heated.

The above-discussed embodiments of the present application will be described further hereinbelow. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

The objects of the present application will become more apparent, when viewed on conjunction with the following drawings in which:

FIG. 1 shows a basic schematic plan view of a clean room filling device for use with the method of the present application;

FIG. 1A shows a more detailed schematic plan view of FIG. 1, of a clean room arrangement utilized for sterilizing and filling containers passing therethrough;

FIG. 2 shows a basic schematic connection diagram of FIG. 1; and

FIG. 2A shows a more detailed schematic diagram of the fluid system shown in FIG. 2, for servicing that clean room arrangement.

### DESCRIPTION OF EMBODIMENT OR EMBODIMENTS

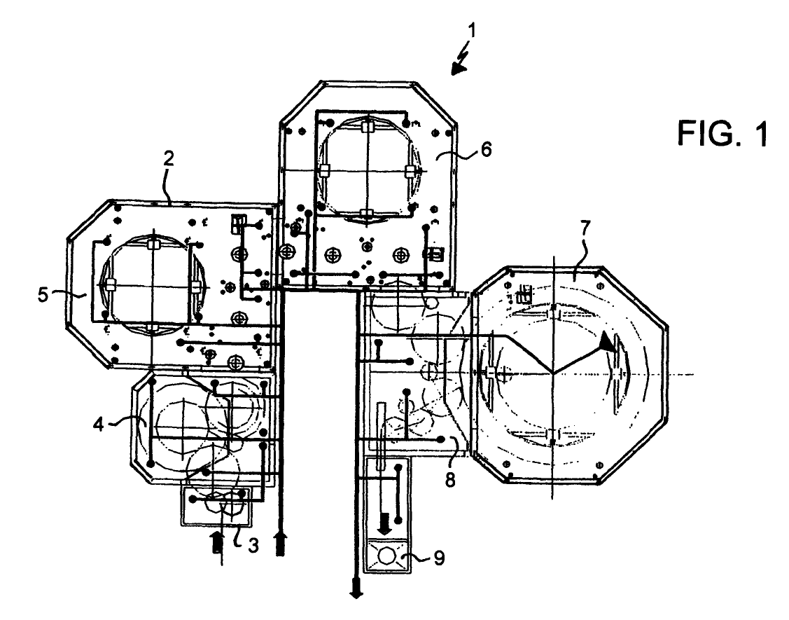

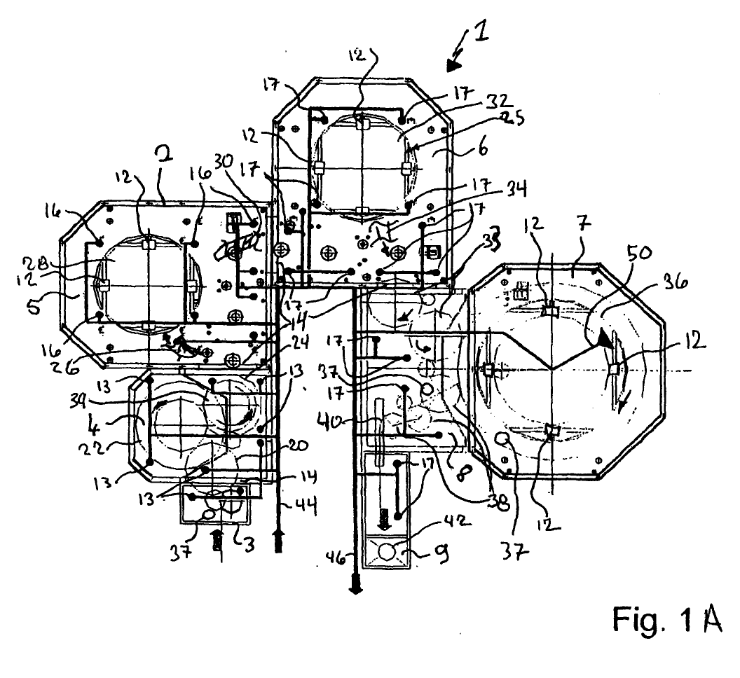

A machine designated in its entirety as 1 for the filling of containers, for example for bottles or cans with beverages, etc., is shown in greater detail in FIGS. 1 and 1A. The complete machine is enclosed in a housing 2, which protects the entire interior from contaminants from the outside so that clean room conditions prevail in the interior.

The machine 1 has a container opening 3, through which the bottles to be filled can gain admission to the interior of the machine. The containers are then sterilized in a sterilization device 4. They move around a circular track together with a plurality of vaporization heads that direct vaporous H₂O₂ into the interior and the upper outer region of the container. Due to the temperature differences between the vaporized H₂O₂ and the wall of the container, the sterilizing agent precipitates and forms a condensation film. The containers are then conveyed into a first activation device 5, in which hot air or hot steam is directed against the outside or the inside of the container. This initiates a decomposition reaction in the deposited H₂O₂, in which it decomposes via a number of intermediate steps. During the reaction, any germs or impurities present are attacked and broken down by the reaction products produced intermittently, until essentially only water and a few residual decomposition products remain.

Because the reaction requires and/or desires some time, once the containers have completed a partial rotation through the first activation device 5, the containers also complete another rotation in a second activation device 6 downstream of the first, so that there is sufficient time for the decomposition reaction to be completed when the end of the second activation device 6 is reached. The bottles then move into the filling device 7, in which the bottles are filled as they move around the machine before they are closed in a closing device 8. They then leave the bottling machine through the container outlet 9.

To maintain the clean room conditions in the interior of the machine 1, the interior must be sterilized periodically. The method according to the present application is performed as follows:

First productive operation of the machine is stopped, in one possible embodiment the supply of bottles is interrupted and the various machines in the interior of the machine 1 are shut down. The cleaning cycle is then started.

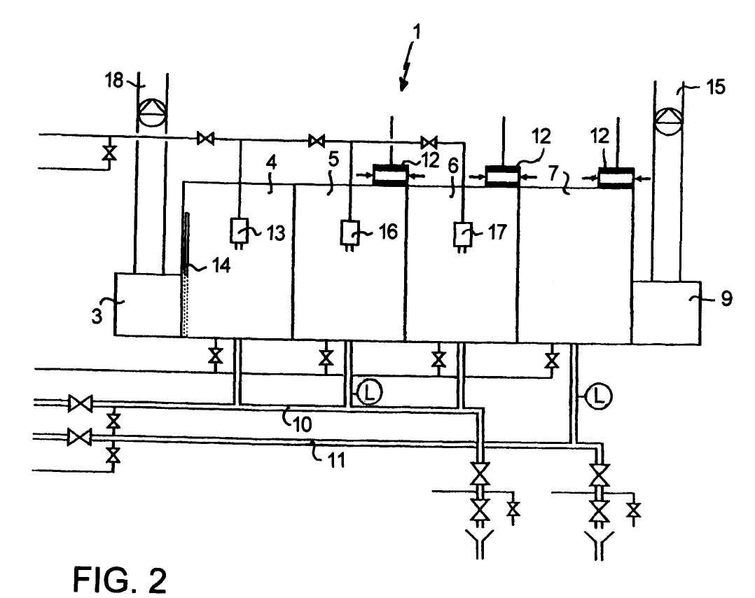

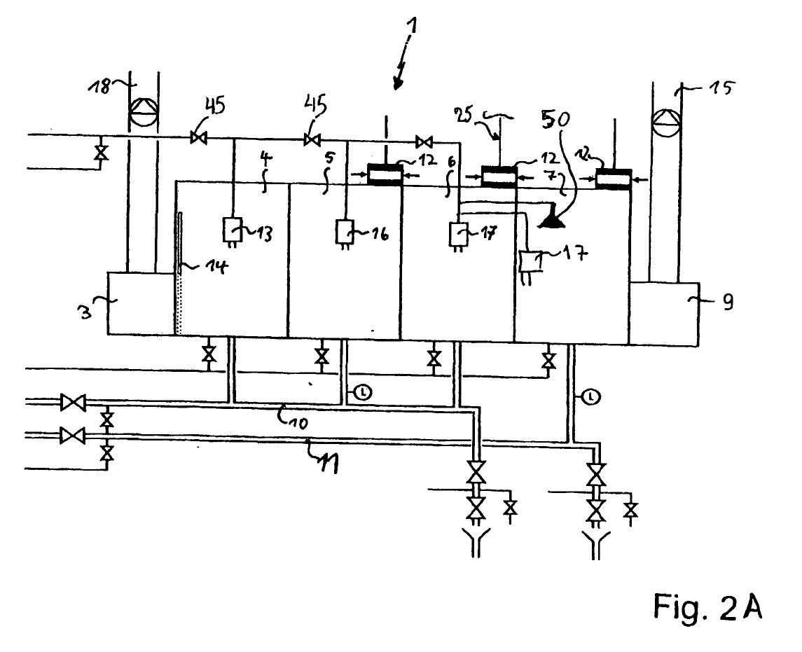

As shown schematically in FIGS. 2 and 2A, there are filtered ventilation devices 12, such as in the form of HEPA filters, for the supply of sterile fresh air to the interior of the machine 1.

In one possible embodiment of the present application, these filters are also cleaned during a cleaning cycle. To this end, these filters are equipped with a device that supplies liquid or vaporous H₂O₂ to the filter layers. In one possible embodiment, the filtered ventilation devices 12 have an integrated heating device, which can then be activated to activate the H₂O₂ in the interior of the filter, as a result of which the sterilization reaction described above can also occur in the interior of the filtered ventilation devices 12.

The sterilization device 4 of the machine 1 is then activated in a second step. Since, as mentioned above, no containers move through the interior of the machine during sterilization of the machine, sterilization heads 13 discharge the vaporous H₂O₂ generated for sterilization directly and unimpeded into the interior of the space encircling the device 4. As described above, the vaporous H₂O₂ precipitates on all surfaces, where it forms a condensation film. To prevent, restrict, and/or minimize the medium/the vaporous H₂O₂ from escaping to the outside, there is a sealing door 14, which is closed at the beginning of the cleaning cycle.

To distribute the vaporous H₂O₂ discharged from the sterilization heads 13 throughout the entire machine 1, any partition doors between the individual machine sections are opened and closed via control action. To provide a controlled air flow in the interior of the machine, an extraction device 15 located at the outlet, for example, can be activated by control action so that air begins to flow through the machine 1, which air flow transports the vaporous H₂O₂ emerging from the sterilization heads 13 through the entire machine 1, where it condenses on each and every surface to form a condensation film as described above.

In one embodiment the two activation devices 5 and 6 can also be used to distribute the gaseous hydrogen peroxide (H₂O₂). Because these devices also include dispensing heads 16 and 17, respectively, for activating the reaction in the containers, vaporous H₂O₂ can be dispensed through these heads during the sterilization cycle rather than the hot air or hot steam dispensed during normal operation.

The lines 10, 11 for the product, for example, are then impinged with a mixture of H₂O₂ and hot steam, so that these are sterilized by the sterilization reaction described above. Some of the decomposition products, such as water and other residues, pass through the piping into the interior of the machine. If there are branch lines, valves, etc., a controller essentially ensures or promotes that all sections of the piping are sequentially supplied with a sufficient amount of the sterilizing mixture.

Once the targeted admission of air and the dispensing of an appropriate amount of the sterilizing medium has produced a sufficient concentration in the interior of the machine and thus a sufficient condensation film has formed on the surfaces in the machine, the reaction must be activated by the addition of a sufficient amount of heat.

The filtered ventilation devices 12 described above are used for this purpose. In this embodiment, these are equipped as described with an integrated heating device and can thus direct an appropriate amount of hot, filtered air into the interior of the machine until the reaction starts. Alternatively, an external heat source can be installed upstream of the filters, which heat source directs an amount of air sufficient for sterilization through the filtered ventilation devices.

After the addition of the appropriate amount of hot air, the described decomposition reaction begins and the precipitated and possibly vaporous H₂O₂ still in the machine decomposes into water and decomposition residues, while any germs and other impurities are broken down and thus the entire machine is sterilized.

Once the reaction has ended, any residues of the vaporous or gaseous sterilization medium are removed from the interior of the machine, whereby it is possible to dry the machine at the same time.

This is done by activating the extraction devices 15 and 18, opening the inlet door 14 and drawing hot air into the interior of the machine through the heated filtered ventilation devices 12 and then extracting it through the extraction devices 15, 18.

In at least one possible embodiment according to the present application, it is possible to feed the hot air into interior of the machine, in one possible embodiment at the approximate center of the process path, and then to extract it in the direction of the two ends of the process path via the extraction devices 15 and 18, in other words in the direction of the inlet 14 and in the direction of the container outlet 9 so that the ingress of germs is further impeded by this procedure.

The machine is now sterilized and can be restarted for productive operation.

The present application is not restricted to the embodiments above and can be modified in numerous ways without straying from the basic principle. The configuration of the machine, for example, is highly variable. The method sequence can also be changed, or a portion of the steps can take place simultaneously or substantially simultaneously. The sterilizing medium used can also be modified as long as it satisfies sterilization performance requirements or desires and is appropriate for the intended use of the machine.

The description above referred only to a vaporous sterilizing medium, for example, but it is obvious to a technician skilled in the art that the above is also applicable to an aerosol sterilizing medium. The sterilizing medium can also be in a state in which the aggregate states vapor and liquid occur together or simultaneously or in constant alternation.

All that is important for the aggregate state of the sterilizing medium is that once it is discharged from its source, such as the sterilization heads 13, into the interior of the clean room, its interior walls are wetted as completely as possible with a condensation or precipitation film.

The present application comprises an aseptic beverage container filling plant for filling containers with an aseptic fluid, the container filling plant comprising a clean room arrangement comprised of a “clustered,” generally circular housing enclosure 35 having a distribution system 25 which maintains the enclosure 35 under positive atmospheric pressure, as represented in FIGS. 1 and 1A, by the controllable fluid distribution system 25, servicing that clustered housing enclosure 35. The distribution system 25, is represented in FIGS. 2 and 2A. The clustered housing enclosure 35, comprises a generally annular array of adjacent, interconnected, segregated and independently operable chambers, 4, 5, 6 and 7, which are in one possible embodiment collectively utilized, as for example, a container/bottle filling machine 1. The container/bottle filling machine 1 has in a first position, a container introduction arrangement 3 in which a series of containers 37, (e.g. bottles) are fed onto a first rotary table 20 through a first inlet door 14.

The inlet door 14 may be of manual operation in one embodiment, or may be automatically opened by, for example, sensors triggered by the containers 37. The first inlet door 14 is openable to an initial container sterilization chamber 4. The sterilization chamber encloses a circular track comprising a second star wheel rotary table and container articulation mechanism 22, for either “batch” or “sequentially” orienting, re-orienting and physically manipulating the containers 37 and transferring the containers 37 through that sterilization chamber 4 onto a third star wheel rotary table 39 within that chamber 4, as the containers 37 are sterilized, as will be described more completely, hereinbelow. Those containers or bottles 37 travel, during sterilization, on their serpentine path within the sterilization chamber 4 on the third rotating table 39 thence to a transport line 26 and may travel through another or second openable and closable chamber-sealing, passageway door 14, and into a first activation chamber 5. Such second door 14 may be of manual operation, or alternatively, be automatically controlled by sensor means, not shown, within the sterilization chamber 4. Those containers 37 are carried therewithin on a first activation chamber star wheel container-manipulating rotary table 28 where the containers 37 may be “batch” or “sequentially” cleansed, prepped, “standards-evaluated”, and then may be advanced onto a bottle transfer line 30 through a third or further openable/closable, sealable door 14 into a second activation chamber 6. The third door 14 may be of manual or automatic operation, in a manner described hereinabove for the other “between-chamber” doors 14. Those containers 37 are thence rotated about a second activation chamber star wheel container-manipulating rotary table 32. The rotary table 32 may include container draining manipulation mechanisms to promote the cleansing and sterilization process. The containers 37 are thence directed to a container/bottle transfer line 34 via a serpentine path comprising two inter-communicating star wheel rotatable-table transfer mechanisms 33. The rotatable transfer mechanisms 33 may in one embodiment, include means for further evaluation of cleansing standards by an container evaluator and automatic failed-container-reject device as part of the transfer mechanisms 33, and thence delivered into the container filling chamber 7. It is noted that some beverage fillings may tolerate a limited amount of treatment fluid in or on the containers and some may not, depending on the characteristics of the fillings.

The containers 37 within the container filling chamber 7 are carried about on a rotary container/bottle support table filling apparatus 36 in which the containers 37 are filled with a “consumptive filling” by a proper container filling mechanism, not shown for clarity of viewing. Those now-filled containers 37 then continue on the rotary filling table 36, and may include in another embodiment, a further examination and pass/fail evaluation through an evaluation mechanism as part of a series of three inter-communicating rotary table transfer modules 38 and thence into a container closing device 8. The individual containers 37 are therein sealed and passed onto a container alignment track 40. The container/bottle alignment track 40 may in a further embodiment, include a label mechanism, to label the successfully-filled, sterilized, non-rejected containers 37, which then pass through a further outlet door 14 and thence through a filled-container discharge port 9, which may in one embodiment, have an excess cleansing fluid drain 42 therein.

A cleansing fluid inlet line 44, represented in FIGS. 1, 1A, 2 and 2A, which feeds a pressurized “treatment fluid” through a set of valving arrangements into first the bottled entry port 3, and also into the sterilization chamber 4. This cleansing fluid supply conduit 44 effects discharge of cleansing fluid through a plurality of discharge heads 13 directed downwardly onto the containers 37, and either sequentially and/or alternately, onto the rotary table arrangements 20, 22 and 24 within that initial sterilization chamber 4 of the cluster housing assembly 35. That fluid cleansing fluid input line 44 through an arrangement of controlled valving means 45, directs cleansing fluid which may in multiple embodiments, be accomplished in timed, temperature and/or a pressure controlled manner, into the first activation chamber 5, through a plurality of discharge heads 16 arranged within the first activation chamber 5, spraying cleansing fluid downwardly onto the containers 37 which are moving on the rotary table 28 in the first activation chamber 5.

In an alternative machine cleansing format, the discharge heads 13, 16, and 17 are utilized to spray cleansing fluid directly onto empty/unloaded rotary tables 22, 24, 28, 30, 32, 33, 34, 36, 38, 40 and 50, and their interlinked associated transfer and container manipulation mechanisms, when it is time and necessary or desired for such mechanism's sterilization/cleaning without any containers in the machine 1, at all.

During such a machine 1 cleansing/sterilization operation, the positive pressure fluid inlet port 44 further directs pressurized fluid through a further controlled set of valves 45 and discharge heads 17, into the second activation chamber 6. That cleansing fluid is directed downwardly onto containers 37 maintained on the second rotary table 32 and onto the rotary table 32 by itself, in the second activation chamber 6 therewithin, when such rotary table 32 and associated mechanism requires cleaning. The dispensing heads 17 controllably dispense cleansing fluid onto the rotary table 32 (and the containers traveling thereon within that entire housing as well, when the machine 1 is utilized to clean the containers 37) in a controlled, container-free, pressure and timed sequence. The cleansing fluid input line 44 further provides a controlled array of discharge heads 17 within a portion of the container filling device 7.

A fluid discharge line 46 comprises the return flow of cleansing fluid from the input line 44 circuit. The fluid system input line 44 also provides a controlled shower spray fill-table washer head 50 arranged in the container filling chamber 7 within the housing 2 of the clean room arrangement 1, to cleanse and sterilize the rotary table mechanism 36 when the table mechanism 36 is not supporting containers being filled.

The first activation chamber 5, the second activation chamber 6 and the container filling chamber 7, are also in fluid communication with one another through a controlled network of pressurizable filter vents 12 represented in FIGS. 1, 1A, 2 and 2A. Those filter vents 12 are arranged to provide pressurized heated, treated, air and/or steam to those centralized first activation chamber 5, the second activation chamber 6 and the container filling chamber 7. That filter vent system 25 provides a positively pressurized gaseous fluid flow within each of those chambers 4, 5, 6 and 7, thence outwardly, through one and/or both of the inlet port 3 and the adjacently aligned product discharge port 9, so as to present a positive pressurized flow of sterilized gaseous fluid, thus denying ingress of contaminants into the housing 2 of the sterilizable clean room arrangement 1 by a constant, or controllably variable, simultaneous, or substantially simultaneous flow of gaseous cleansing fluid out of both the container ingress port 3 and the container egress port 9, either during the container cleansing and filling process, or during the cleansing/sterilization of the machine 1, by itself, without any containers traveling therewithin, thus eliminating, restricting, and/or minimizing contaminant entry into the clean room container filling machine arrangement 1 under any and all operating conditions.

The present application may also include a method for the sterilization of clean rooms for the handling and/or filling of containers, whereby at least one device for the sterilization of containers 4 using a vaporous or aerosol sterilizing medium is located in the interior of the clean rooms, and wherein the vaporous or aerosol sterilizing medium is dispensed from the device for the sterilization of containers 4 into the environment, the dispensed vaporous or aerosol sterilizing medium is directed into the clean room by means of targeted air routing, after which a precipitation or condensation film is formed inside the clean room from the sterilizing medium; and wherein the sterilizing medium and/or the precipitation or condensation film is activated and the sterilization reaction started by the addition of an activation medium; wherein the vaporous H₂O₂ is used as the sterilizing medium; and wherein heated air and/or gas and/or steam is used as the activation medium; and wherein the pipelines present are sterilized using the same sterilizing and/or activation medium; and wherein at least one container sterilization device 4 and/or one container filling device 7 is used inside the clean room; and wherein at least one container activation device 5 and/or one closing device 8 is used inside the clean room; and wherein the vaporous sterilizing medium is also dispensed to the environment from the activation device 5; and wherein controlled ventilation devices 12 and/or controlled air inlets and/or controlled air outlets and/or controlled doors 14 are used for targeted air routing; and wherein the residues of the sterilizing medium and/or the decomposition products in the clean room are extracted by means of at least one extraction device 15, 18; and wherein heated air or heated gas is admitted, in one possible embodiment in the center of the handling path, to extract residues of the sterilizing medium and/or decomposition products in the clean room and/or to dry the clean room, which air or gas is extracted in the direction of the container inlet 3 and/or in the direction of the container outlet 9; including a cleaning device for the sterilization of clean rooms for the handling and/or filling of containers using the method according to the present application.

A method for the sterilization of clean rooms for the handling and/or filling of containers is to be provided that enables effective sterilization of the interior of the clean rooms without having to install additional costly and complex cleaning systems.

To achieve this, the vaporous or aerosol sterilizing medium is dispensed from a device for the sterilization of containers 4 into the environment, the dispensed vaporous or aerosol sterilizing medium is directed into the clean room by means of targeted air routing, after which a condensation film is formed inside the clean room from the sterilizing medium.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

All of the patents, patent applications or patent publications, which were cited in the International Search Report dated Oct. 31, 2007, and/or cited elsewhere are hereby incorporated by reference as if set forth in their entirety herein as follows: EP 0 243 073, having the title “CLEAN AIR SYSTEM,” published on Oct. 28, 1987; JP11 208782, having the following English translation of the Japanese title “METHOD AND APPARATUS FOR STERILIZING FILLING MACHINE,” published on Aug. 3, 1999; DE 44 25 219, having the following English translation of the German title “CLEANING AND STERILISING BOTTLE RINSING MACHINE,” published on Jan. 18, 1996; U.S. Pat. No. 5,173,259, having the title “STERILIZATION METHOD FOR A PACKING MACHINE THAT USES LIQUID DISINFECTANT,” published on Dec. 22, 1992; and JP 08 091490, issued to Mitsubishi Heavy Ind Ltd., published on Apr. 9. 1996.

Federal Republic of Germany Patent Application No. DE 10 2006 036 476.7, filed on Aug. 4, 2006, and its corresponding International Patent Application No. PCT/2007/006806, filed on Aug. 1, 2007, having WIPO Publication No. WO 2008/014992 and inventor Daryoush SANGI are hereby incorporated by reference as if set forth in their entirety herein.

All of the patents, patent applications or patent publications, which were cited in the German Office Action dated Apr. 4, 2007, and/or cited elsewhere are hereby incorporated by reference as if set forth in their entirety herein as follows: DE 37 01 079, having the following English translation of the German title “METHOD FOR DISINFECTING PACKAGING CONTAINERS,” published on Jul. 28, 1988; and DE 196 42 987, having the following German title “VERFAHREN UND VORRICHTUNG ZUM STERILISIEREN UND BEFÜLLEN VON VERPACKUNGSBEHÄLTERN,” published on Apr. 23, 1998.

The patents, patent applications, and patent publication listed above, beginning on line 946 on page 46 in the paragraph with the phrase: “All of the patents, patent applications or patent publications, which were cited in the International Search Report . . . ” and ending on line 976 on page 47 in the paragraph with the phrase: “ . . . published on Apr. 23, 1998” are herein incorporated by reference as if set forth in their entirety. The purpose of incorporating U.S. patents, Foreign patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. 10 2006 036 475.9, filed on Aug. 4, 2006, having inventors Daryoush SANGI and Thomas HEROLD, and DE-OS10 2006 036 475.9 and DE-PS10 2006 036 475.9, and International Application No. PCT/EP2007/006805, filed on Aug. 1, 2007, having WIPO Publication No. WO 2008/014991 and inventors Daryoush SANGI and Thomas HEROLD, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the Foreign equivalent patent application PCT/EP2007/006805 and German Patent Application 10 2006 036 475.9 is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2007/006805 and DE 10 2006 036 475.9 from which this patent application claims priority which do not have to do with the correction of the translation in this patent application are not to be included in this patent application in the incorporation by reference.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.
   Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

What is claimed is:

1. A process for cleaning an aseptic beverage bottle filling plant and aseptically filling beverage bottles therein, the operative process comprising:
arranging a generally circular array of chambers into a series of bottle treatment mechanisms, and forming the beverage bottle filling plant comprising a cleaning chamber, a first activation chamber, a second activation chamber and a bottle filling chamber, the cleaning chamber comprising a bottle entry port and the bottle filling chamber having a bottle discharge port;
connecting the chambers with bottle transport apparatus for moving treated bottles therebetween;
installing a plurality of fluid discharge heads within more than one of the chambers, and connecting the discharge heads to a fluid supply circuit;
installing a plurality of vent units for the discharge of a second fluid within the activation chambers and the bottle filling chamber;
installing partition doors within the beverage bottle filling plant configured to control the flow of any treatment fluid and any activation fluid within the chambers of the filling plant;
applying a treatment fluid within the cleaning chamber through the fluid discharge heads therein;
distributing treatment fluid through the generally circular array of chambers by activating a first extraction device configured to move the treatment fluid through all the chambers;
delivering an activation gas through the vent units at a general mid-point of a process path through which the bottles travel within the bottle filling plant;
activating the treatment fluid dispersed onto the bottle treatment mechanisms;
activating a second gas extraction device to permit the movement of the gas and activation fluid out both an open first and an open second port which first and second ports are generally adjacent one another, to thus permit the bottle filling plant to be cleansed by controlled fluid routing, and thus exhausting the activation fluid and the gas from both the first and second port through the shortest most direct routes out of the beverage bottle filling plant, to impede entry of germs into the filling plant, therefore minimizing travel distance and maintaining maximum effectiveness of treatment activity of treatment gases and thus readying the beverage bottle filling plant for a bottle filling cycle;
delivering a plurality of empty bottles into the first port in the first or cleaning chamber;
directing a treatment fluid onto the empty bottles within the first or cleaning chamber;
moving the empty bottles through the bottle process path for their subsequent activation treatment in the second chamber and the third chamber;
applying an activation fluid onto the empty bottles during their travel on the process path through the second and third activation chambers;
directing the now-sterilized empty bottles into the filling chamber for subsequent filling of the bottles with a consumable beverage;
filling the empty bottles located within the filling chamber with a consumable beverage;
closing the filled bottles and directing those bottles out of the filling plant on a filled-bottle discharge track; and
discharging the now-filled bottles out of the beverage bottle filling plant through the second port, thus completing the generally circular route of the beverage bottles through the beverage bottle filling plant.

2. The process for operating a beverage bottle filling plant according to claim 1, wherein:
said first or cleaning chamber comprises a sterilization chamber; and
said method further comprises introducing treatment fluid through the discharge heads within the activation chambers and the bottle filling chamber, so as to provide the treatment fluid for cleansing the bottle treatment mechanisms therein.

3. The process for operating a beverage bottle filling plant according to claim 2, wherein the treatment fluid comprises hydrogen peroxide.

4. The process for operating a beverage bottle filling plant according to claim 3, wherein:
the gas delivered through the vent units to activate the treatment fluid is a heated gas; and
the gas delivered to the filling chamber is a purified gas.

5. An apparatus for performing the process of operating an aseptic beverage bottle filling plant, configured to perform the steps according to claim 1, the apparatus comprising:
an isolated generally circular array of chambers into a series of bottle treatment mechanisms, for the forming of the beverage bottle filling plant comprising a sterilization chamber, a first activation chamber, a second activation chamber and a bottle filling chamber, the sterilization chamber comprising a bottle entry port and the bottle filling chamber having a bottle discharge port, the chambers being connected with bottle transport lines for moving treated bottles therebetween;
a plurality of fluid discharge heads arranged within more than one of the chambers, the discharge heads being connected to a fluid supply circuit;
a plurality of vent units arranged for the discharge of a second fluid within the activation chambers and the bottle filling chamber;
an arrangement of partition doors within the beverage bottle filling plant configured to control the flow of any treatment fluid and any activation fluid within the chambers of the filling plant;
a treatment fluid discharged within the sterilization chamber through the fluid discharge heads therein, the treatment fluid distributed through the generally circular array of chambers by activating a first extraction device configured to move the treatment fluid through all the chambers;
a gas delivered through the vent units at a general mid-point of a process path through which the bottles travel within the bottle filling plant, and wherein the treatment fluid is dispersed onto the bottle treatment mechanisms;
a second gas extraction device being arranged to move the gas and activation fluid out an open first and an open second port which ports are generally adjacent one another, to thus permit the bottle filling plant to be cleansed by controlled fluid routing, and thus exhausting the activation fluid and the gas from both the first and second port through the shortest most direct routes out of the beverage bottle filling plant to impede entry of germs into the filling plant, therefore minimizing travel distance of treatment gases thus readying the beverage bottle filling plant for a bottle filling cycle, and wherein a plurality of empty bottles are arranged for delivery into the first port in the first or sterilization chamber;
a treatment fluid for discharge onto the empty bottles within the first or sterilization chamber, wherein the empty bottles are moved through the bottle process path for their subsequent activation treatment in the second chamber and the third chamber;
an activation fluid application onto the empty bottles during their travel on the process path through the second and third activation chambers; wherein the now-sterilized empty bottles are directed into the filling chamber for subsequent filling of the bottles with a consumable beverage, and filling the empty bottles located within the filling chamber with a consumable beverage;
a closing apparatus for the filled bottles and a directing apparatus for those bottles out of the filling plant on a filled-bottle discharge track; and
a discharge apparatus for moving the now-filled bottles out of the beverage bottle filling plant through the second port, thus completing the generally circular route of the bottles through the beverage bottle filling plant.

6. The apparatus for performing the process of operating an aseptic beverage bottle filling plant according to claim 5, including: a heating apparatus arranged within the vent units, the heating apparatus arranged to controllably heat fluid passing therethrough.

7. A method for operating an aseptic beverage container filling plant, comprising:
connecting an array of chambers into a series of container treatment mechanisms, and forming the beverage container filling plant from those chambers which together comprise a sterilization chamber, a first activation chamber, a second activation chamber and a container filling chamber, the sterilization chamber comprising a container entry port and the container filling chamber having a container discharge port;
connecting the chambers with container transport apparatus for moving treated containers therebetween;
installing a plurality of fluid discharge heads within more than one of the chambers, and connecting the discharge heads to a fluid supply circuit;
installing a plurality of vent units for the discharge of a second fluid within the activation chambers and the container filling chamber;
installing partition doors within the beverage container filling plant, the doors configured to control the flow of any treatment fluid and any activation fluid within the chambers of the filling plant;
applying a treatment fluid within the sterilization chamber through the fluid discharge heads therein;
distributing treatment fluid through the array of chambers by activating a first extraction device configured to move the treatment fluid through all the chambers;
delivering an activation gas through the vent units located adjacent the process path through which the containers travel within the container filling plant;
activating the treatment fluid dispersed onto the container treatment mechanisms;
activating a second gas extraction device to permit the movement of the gas and activation fluid out multiple ports which ports are generally adjacent one another, to thus permit the bottle filling plant to be cleansed by controlled fluid routing, and thus removing the activation fluid and the gas from both the multiple ports through the shortest most direct routes out of the beverage container filling plant, to impede entry of germs into the filling plant, therefore minimizing travel distance and maintaining maximum effectiveness of treatment activity of treatment gases and thus readying the beverage container filling plant for a container filling cycle;
delivering a plurality of empty containers into the first port in the first or sterilization chamber;
directing a treatment fluid onto the empty containers within the first or sterilization chamber;
moving the empty containers through the container process path for their subsequent activation treatment in the second chamber and the third chamber;
applying an activation fluid onto the empty containers during their travel on the process path through the second and third activation chambers;
directing the now-sterilized empty containers into the filling chamber for subsequent filling of the containers with a consumable beverage;
filling the empty containers located within the filling chamber with a consumable beverage;
closing the filled containers and directing those containers out of the filling plant on a filled-container discharge track; and
discharging the now-filled containers out of the beverage container filling plant through the second port, thus completing the process path route of the beverage containers through the beverage container filling plant.

8. The method according to claim 7, wherein the process path of the containers within the beverage processing plant is generally of circular configuration.

9. The method according to claim 8, wherein the activation gas is heated.

10. The method according to claim 8, wherein:
the method for the sterilization of the chambers for the handling and/or filling and the sterilization of the containers uses a vaporous or aerosol sterilizing medium as the treatment fluid to be distributed from the vent units located in the interior of the chambers;
the vaporous or aerosol sterilizing medium is dispensed into the environment, the dispensed vaporous or aerosol sterilizing medium being directed into the chambers by use of targeted air routing, after which a precipitation or condensation film is formed inside the chambers from the sterilizing medium;
the sterilizing medium and/or the precipitation or condensation film is activated and a sterilization reaction started by the addition of the activation fluid;
a vaporous H₂O₂ is used as the sterilizing fluid;
heated air and/or gas and/or steam is used as the activation fluid;
the fluid supply circuit present is also sterilized using the same sterilizing and/or activation fluid;
at least one container sterilization chamber 4 and/or one container filling device 7 is used inside the plant, and wherein at least one container activation chamber 5 and/or one closing device 8 is used inside the plant;
the vaporous sterilizing fluid is also dispensed to the environment from the activation chamber 5;
the vents units 12 and/or the fluid discharge heads and/or the partition doors 14 are used for controlled targeted air routing;
the residues of the sterilizing medium and/or the decomposition products in the chambers are extracted by use of at least one extraction device 15, 18;
heated air or heated gas is admitted, preferably in the center of the process path, to extract the residues of the sterilizing medium and/or the decomposition products in any chamber in which air or gas is extracted in the direction of the container inlet 3 and/or in the direction of the container outlet 9.

11. A process for establishing and operating a substantially aseptic container filling plant, the process comprising:
arranging a generally non-linear array of connected chambers into a series of container treatment mechanisms, and forming the container filling plant comprising a sterilization chamber, a first activation chamber, a second activation chamber and a container filling chamber, the sterilization chamber comprising a container entry port and the container filling chamber having a container discharge port;
arranging a container transport apparatus between the connected chambers, for moving treated containers therebetween;
installing a plurality of fluid discharge heads within more than one of the chambers, and connecting the discharge heads to a fluid supply circuit;
installing a plurality of vent units for the discharge of a second fluid within the activation chambers and the container filling chamber;
installing partition doors within the container filling plant configured to control the flow of any treatment fluid and any activation fluid within the chambers of the filling plant;
applying a treatment fluid within the sterilization chamber through the fluid discharge heads therein;
distributing treatment fluid through the generally non-linear array of chambers by activating a first extraction device configured to move the treatment fluid through all the chambers;
delivering an activation gas through the vent units in the non-linear array of chambers through which the containers travel within the container filling plant;
activating the treatment fluid dispersed onto the container treatment mechanisms;
activating a second gas extraction device to permit a dual path evacuation of the gas and activation fluid from out both an open first and an open second port, to thus permit the bottle filling plant to be cleansed by controlled fluid routing, and thus exhausting the activation fluid and the gas from both the first and second port through the shortest most direct routes out of the beverage container filling plant, to impede entry of germs into the filling plant, therefore minimizing travel distance and maintaining maximum effectiveness of treatment activity of treatment gases and thus readying the container filling plant for a container filling cycle;
delivering a plurality of empty containers into the first port in the first or sterilization chamber;
directing a treatment fluid onto the empty containers within the first or sterilization chamber;
moving the empty containers through the container process path for their subsequent activation treatment in the second chamber and the third chamber;
applying an activation fluid onto the empty containers during their travel on the process path through the second and third activation chambers;
directing the now-sterilized empty containers into the filling chamber for subsequent filling of the containers;
filling the empty containers located within the filling chamber;
closing the filled containers and directing those containers out of the filling plant on a filled-container discharge track; and
discharging the now-filled containers out of the container filling plant through the second port, thus completing the generally circular route of the containers through the beverage container filling plant.

12. An apparatus configured to perform the process according to claim 11, the apparatus comprising:
a generally non-linear array of connected chambers comprising a series of container treatment mechanisms wherein the container filling plant comprises a sterilization chamber, a first activation chamber, a second activation chamber and a container filling chamber, the sterilization chamber comprises a container entry port and the container filling chamber has a container discharge port;
a container transport apparatus arranged between the connected chambers, for moving treated containers therebetween;
a plurality of fluid discharge heads within more than one of the chambers, with the discharge heads connected to a fluid supply circuit;
a plurality of vent units for the discharge of a second fluid within the activation chambers and the container filling chamber;
an arrangement of partition doors within the container filling plant configured to control the flow of any treatment fluid and any activation fluid within the chambers of the filling plant;
an arrangement configured to disperse a treatment fluid within the sterilization chamber through the fluid discharge heads therein;
the treatment fluid dispersable through the generally non-linear array of chambers through an activatable first extraction device configured to move the treatment fluid through all the chambers;
an arrangement configured to deliver an activation gas through the vent units in the non-linear array of chambers through which the containers travel within the container filling plant, and whereby the treatment fluid is dispersed onto the container treatment mechanisms;
an activatable second gas extraction device to permit a dual path evacuation of the gas and activation fluid from out both an open first and an open second port, to thus permit the bottle filling plant to be cleansed by controlled fluid routing, and thus for the exhaust of the activation fluid and the gas from both the first and second port through the shortest most direct routes out of the beverage container filling plant, to impede entry of germs into the filling plant, to therefore minimize travel distance and to maintain maximum effectiveness of treatment activity of treatment gases and so the container filling plant is ready for a container filling cycle; a plurality of empty containers receivable in the first port in the first or sterilization chamber;
an arrangement configured to apply a treatment fluid onto the empty containers within the first or sterilization chamber;
an apparatus configured to move the empty containers through the container process path for their subsequent activation treatment in the second chamber and the third chamber;
an arrangement configured to apply an activation fluid onto the empty containers during their travel on the process path through the second and third activation chambers;
an apparatus configured to direct sterilized empty containers into the filling chamber for subsequent filling of the containers;
an apparatus to fill the empty containers located within the filling chamber;
an apparatus to close the filled containers and direct those containers out of the filling plant on a filled-container discharge track; and
apparatus to discharge filled containers out of the container filling plant through the second port, to thus establish the generally circular route of the containers through the beverage container filling plant.

13. The process according to claim 11, wherein the treatment fluid comprises hydrogen peroxide.

14. The process for operating a beverage container filling plant according to claim 11, wherein the gas delivered through the vent units to activate the treatment fluid is a heated gas.

15. The process for operating a beverage container filling plant according to claim 13, wherein the gas delivered to the filling chamber is a purified gas.

16. The process according to claim 14, wherein the filling plant has been through the cleansing process prior to the filling plant receiving empty beverage containers.

17. The process according to claim 11, wherein the activation gas is delivered through the vent units at a mid-point in the non-linear array of chambers.

18. The process according to claim 11, wherein the containers are filled with a substantially aseptic beverage.

19. A control device for controlling the operation of the container filling plant according to the process of claim 11.

20. A method for controlling the operation of the container filling plant, according to claim 11, by arranging a control device to regulate the flow of fluids, gases and containers within the chambers of the plant.
